(19) United States
(12) Patent Application Publication
(10) Pub. No.: US 2006/0241691 A1
(43) Pub. Date: Oct. 26, 2006

(54) Medical treatment method and device utilizing magnetic elements

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/386,506

(22) Filed: Mar. 22, 2006

Related U.S. Application Data
(60) Provisional application No. 60/670,511, filed on Apr. 12, 2005.

(51) Int. Cl. A61B 17/08

(52) U.S. Cl. 606/215

(57) ABSTRACT

In a medical treatment method, magnetic elements are placed into contact with organic tissues of a patient. The magnetic elements are disposed, upon deployment, on opposite sides of tissues or a hole to be closed or collapsed. Owing to magnetic attraction between the magnetic elements, the organic tissues of the patient are held together to constrict tissues or to close or collapse a wound or vessel.

### CROSS-REFERENCE TO RELATED APPLICATION

This application claims the benefit of U.S. Provisional Patent Application No. 60/670,511 filed Apr. 12, 2005.

### BACKGROUND OF THE INVENTION

This invention relates to a medical treatment method and an associated device. The method may be used in the treatment of such conditions as esophageal varices, hemorrhoids, tumors, and wounds or holes particularly in internal tissues.

Many medical conditions involve swollen tissues which attain such a size as to inhibit or interfere with one or more necessary physiological functions. An example of such swollen tissues are varices of the digestive tract. Gastric and esophageal varices are a devastating complication of portal hypertension. To treat such bleeding varices, it is necessary at times to use a long tube with two inflatable balloons at a distal end, known as a “Blakemore Tube.” In using this device to stop the flow of blood in the stomach, the tube is blindly inserted into the esophagus until it is believed that the most distal of the two balloons is located in the patient's stomach. That balloon is then inflated and the tube placed in tension (e.g., via attachment to a weight outside of the patient) to pull the inflated balloon against the stomach wall at the gastroesophageal junction. In the event that bleeding esophageal varices are to be treated, the relatively proximal balloon is also inflated.

The rate of complications in the use of the Blakemore tube is immense. The complications result mainly from poor placement or slippage of the tube. In addition, the relatively proximal balloon sometimes erodes into the esophagus, causing bleeding, perforation and necrosis of the esophagus.

Another kind of swollen internal tissues, namely, hemorrhoids, are located at the downstream end of the digestive tract. Hemorrhoids are a common malady which causes substantial pain and suffering to millions of people. The best conventional treatment for this affliction is a soaking of the hemorrhoidal tissues in a hypertonic bath, such as a solution of Epsom salts. However, this treatment is not especially effective. A need exists for a more convenient and yet effective treatment for hemorrhoids.

Certain cancers represent yet another kind of swollen tissues. Once cancer has reached the tumor stage, where lumps of cancerous tissues are detectable either directly through touch and vision or indirectly with the aid of MRI and CAT scanners, the principal treatment is surgical. The victim is operated on and the tumor cut out of the body. Frequently, the location and size of the tumor are such that surgical removal results in a severe impairment to the patient's body and lifestyle. For example, surgical removal of a large tumor in a femur frequently results in an amputation.

The operations for surgically removing tumors are nearly universally open incision type operations. These operations are naturally debilitating and require extensive post surgical care. For these reasons, the costs of conventional open incision surgery are enormous.

Although minimally invasive procedures such as laparoscopic or thoracoscopic surgery have increased at geometric rates in frequency of performance, minimally invasive surgery for the treatment of cancer has not been employed. Of course, other kinds of minimally invasive techniques such as chemotherapy and radiation treatment are widely practiced. However, these techniques have substantial debilitating side effects. Patients must suffer significantly in virtually every case.

Nevertheless, minimally invasive techniques are the future of medicine. Patient trauma and hospitalization time are reduced. In addition, costs and expenses are decreased.

Internal wounds such as perforations of internal organs are also generally treated by open surgery. Such wounds may be the result of traumas. A blunt trauma may cause a rupture, for instance, of the spleen and consequent internal bleeding.

### OBJECTS OF THE INVENTION

An object of the present invention is to provide a method for closing open wounds and incisions.

A further object of the present invention is to provide such a method that may be used in endoscopic, laparoscopic, and other types of minimally invasive surgery such as a trans-organ type of surgery disclosed in U.S. Pat. Nos. 5,297,536 and 5,458,131.

An additional object of the present invention is to provide medical surgical devices for use in closing open wounds and incisions.

These and other objects of the present invention will be apparent from the drawings and descriptions herein. It is to be noted that any one of the above objects may be attained in one or more embodiment of the invention disclosed herein. No one embodiment need attain all of the objects of the invention.

### SUMMARY OF THE INVENTION

U.S. Pat. No. 6,730,014 discloses surgical closure or approximation methodology that involves the injection of magnetic particles into organic tissues on opposite sides of a feature to be closed. The present invention is directed to improvements and enhancements to the method and apparatus disclosed in U.S. Pat. No. 6,730,014.

A medical treatment method in accordance with the present invention comprises placing at least one first magnetic element in contact with first organic tissues of a patient on one side of a feature to be closed or collapsed, placing at least one second magnetic element in contact with second organic tissues of the patient on an opposite side of the feature to be closed or collapsed, and by virtue of a magnetic attraction between the first magnetic element and the second magnetic element, holding the first and the second organic tissues of the patient together to close or collapse the feature.

The placing of the first magnetic element in contact with the first organic tissues may include placing the first magnetic element into contact with and along an external surface of the first organic tissues, such that the first magnetic element remains outside of the first organic tissues. Similarly, the placing of the second magnetic element in contact with the second organic tissues may include placing the second magnetic element into contact with and along an external surface of the second organic tissues, such that the second magnetic element remains outside of the second organic tissues. Alternatively, where the magnetic elements are particles, the magnetic elements may be injected into the tissues of the patient.

Pursuant to another feature of the present invention, at least one of the first magnetic element and the second magnetic element takes the form of an elongate strip. In that case, the placing of the one magnetic element in contact with the respective organic tissues including placing the strip along the respective organic tissues.

The elongate strip may be provided along one surface with a plurality of coupling elements taken from the group consisting of barbs, hooks, and prongs. In that case, the method further comprises inserting the coupling elements into the organic tissues to anchor the strip to the tissues.

Pursuant to a further feature of the present invention, at least one of the magnetic elements includes a clip. In that case, the placing of that magnetic element in contact with organic tissues includes actuating the clip to grasp the organic tissues. Where the feature is a wound or incision having a pair of edges or lips and where the clip has a pair of clamping elements taken from the group consisting of legs, prongs, and arms, the actuating of the clip includes inserting one of the clamping elements of the clip through the wound or incision.

Where the first magnetic element and the second magnetic element are parts of a single clamp, the first magnetic element and the second magnetic element being hingedly connected to one another, the method further comprises pivoting the first magnetic element and the second magnetic element relative to one another to close the clamp on the first organic tissues and the second organic tissues, thereby closing the feature.

The placing of the first magnetic element and the second magnetic element may include inserting an endoscope into the patient and ejecting the first magnetic element and the second magnetic element from a working channel of the endoscope. The working channel may be part of a sheath which surrounds the endoscope.

A medical treatment device in accordance with the present invention comprises an elongate strip of magnetic material provided along a major face or surface with a plurality of coupling elements taken from the group consisting of barbs, prongs and hooks.

Where the strip is one of two wound-closure strips provided in a medical treatment kit, the other of the wound-closure strips is made of magnetic material, and may also be provided along a major face or surface with a plurality of coupling elements taken from the group consisting of barbs, prongs and hooks.

Another embodiment of a medical treatment device in accordance with the present invention comprises a clamp and a magnetic element attached to the clamp. The clamp may include a pair of jaws hingedly secured to one another, the magnetic element being connected to at least one of the jaws.

The present invention may find application in closing or constricting a blood vessel. Other tissues about the blood vessel may also be clamped or collapsed owing to the magnetic attraction between the injected elements. For instance, where the blood vessel is in esophageal varices, the holding or drawing of the organic tissues of the patient together includes a constricting or reducing of the varices. Thus, in one simple procedure, the esophagus is opened and the blood vessels in the varices are closed off, preventing bleeding into the digestive tract.

A procedure for constricting swollen internal tissues in accordance with the present invention is preferably executed in a minimally invasive manner. Thus, where the swollen target tissues are esophageal varices, an endoscope is inserted into the patient's esophagus and the magnetic elements are ejected from a biopsy channel of the endoscope. The optics of the endoscope are used to visually detect the varices and select a point of application or deployment on the varices of the magnetic elements.

Where target swollen tissues are a hemorrhoid, the drawing and holding of the organic tissues of the patient together result in a size reduction of the hemorrhoid, as well as a constricting of one or more blood vessels of the hemorrhoid.

The organic tissues to which the magnetic elements are applied may be a tumor. In that case, the drawing together of the tissues entails an interrupting of a blood supply of the tumor.

The attraction between the magnetic elements serves to at least partially collapse the blood vessels which feed the tumor. In many cases, the magnetic elements may be applied via a minimally invasive procedure to tissues containing a tumor. Where the magnetic elements are particles such as iron filings, a needle may be used to deploy the magnetic elements.

In general, where the target tissues, i.e., the tissues to which the magnetic elements are applied, are swollen tissues such as varices, a tumor, or hemorrhoids, it is not necessary to identify and locate particular blood vessels which are to be closed or collapsed. Instead, the magnetic elements are injected into the target tissues in such numbers and with such a density that blood vessels located in the target tissues are naturally constricted by the movement of the injected magnetic elements under the magnetic attractive forces.

Where the target is an identifiable wound or opening, a permanent magnet is placed into contact with tissues on one side of the wound or opening, while another permanent magnet or one or more magnetizable elements (generally metallic) are disposed on contact with organic tissues on an opposite side of the wound or opening. The magnetic attraction results in a constricting of the tissues and a closure of the wound or opening.

Accordingly, it is contemplated that at least one of the magnetic elements is a permanent magnet. The other magnetic elements may include one or more permanently magnetized particles, as well as one or more magnetizable particles. The magnetizable particles may be made of a metal such as iron or steel or may be made of a polymeric material in which magnetic atoms are embedded. Where the magnetic elements are injected into the tissues of the patient, the magnetic elements may be each formed at one end with a point for facilitating entry into the target tissues and are preferably of a suitable size for exerting a compressive force on the target tissues. In some case, metal filings may be used. Filings generally have sharp points or edges facilitating injection into organic tissues.

It is to be noted that where the magnetic elements or particles are injected into digestive tract tissues, a subsequent dislodgement of the particles merely results in the particles' being flushed from the body with excreted materials.

The present invention provides, inter alia, a method for treating swollen tissues including, but not limited to, hemorrhoidal tissues, esophageal or gastric varices, and tumors. This method is less invasive and less expensive than conventional open-incision surgical techniques. The present method may be used in endoscopic, laparoscopic, and other types of minimally invasive surgery such as the trans-organ procedures disclosed in U.S. Pat. Nos. 5,297,536 and 5,458,131.

A medical treatment kit in accordance with the present invention comprises at least one first magnetic element disposable in contact with first organic tissues of a patient on one side of a feature to be closed or collapsed and at least one second magnetic element disposable in contact with second organic tissues of the patient on an opposite side of the feature to be closed or collapsed. The magnetic elements and have a magnetic attraction to one another sufficient to hold the first and the second organic tissues together to close or collapse the feature. The first magnetic element are disposable in contact with and along an external surface of the first organic tissues, such that the first magnetic element remains outside of the first organic tissues, while the second magnetic element are disposable in contact with and along an external surface of the second organic tissues, such that the second magnetic element remains outside of the second organic tissues.

### BRIEF DESCRIPTION OF THE DRAWINGS

FIGS. 1A-1C are schematic cross-sectional views of an esophagus with varices, showing successive steps in an endoscopic procedure for reducing the varices in accordance with the present invention.

FIGS. 2A and 2B are schematic cross-sectional views of an anus with hemorrhoids, showing successive steps in an endoscopic procedure for shrinking the hemorrhoids in accordance with the present invention.

FIG. 3 is a schematic partial cross-sectional view of a patient's abdomen, showing a step in a laparoscopic procedure for destroying a liver tumor in accordance with the present invention.

FIG. 4 is a schematic view of a tumor, showing a step in an intravascular procedure for destroying the tumor in accordance with the invention.

FIG. 5 is a schematic longitudinal cross-sectional view of a tubular instrument in accordance with the present invention, for performing a medical treatment method pursuant to the invention.

FIG. 6 is a schematic longitudinal cross-sectional view of another tubular instrument in accordance with the present invention, for performing a medical treatment method pursuant to the invention.

FIG. 7 is a schematic longitudinal cross-sectional view of a further tubular instrument in accordance with the present invention, for performing a medical treatment method pursuant to the invention.

FIG. 8 is a schematic longitudinal cross-sectional view of yet another tubular instrument in accordance with the present invention, for performing a medical treatment method pursuant to the invention.

FIG. 9 is a schematic perspective view of an internal organ such as a spleen, showing a bleeding rupture in the organ.

FIG. 10 is a schematic partial perspective view, on a larger scale, of the ruptured organ of FIG. 9, showing an intravascularly implemented procedure for arresting blood flow in accordance with the present invention.

FIGS. 11A and 11B are schematic perspective views of a wound, showing successive steps in a wound-closure method in accordance with the present invention.

FIGS. 12A through 12D are schematic side elevational views of a colon, partially broken away to show successive steps in an endoscopically implemented hole-closure procedure in accordance with the present invention.

FIGS. 13A through 13C are schematic side elevational views similar to FIGS. 12A-12D, showing successive steps in an alternative hole-closure procedure in accordance with the present invention.

FIGS. 14A through 14C are schematic perspective views showing another procedure for closing a wound in accordance with the present invention.

FIGS. 15A and 15B are schematic perspective views, with a cross-section of an internal organ, showing a further procedure for closing a wound or incision in accordance with the present invention, also showing a surgical closure device in accordance with the present invention.

FIGS. 16A and 16B are schematic perspective views showing yet another procedure for closing a wound or incision in accordance with the present invention, also showing a surgical closure device in accordance with the present invention.

FIG. 17 is a schematic perspective view of an instrument and surgical closure device, together with a cross-sectional of an internal organ, showing a procedure in accordance with the present invention.

### DETAILED DESCRIPTION

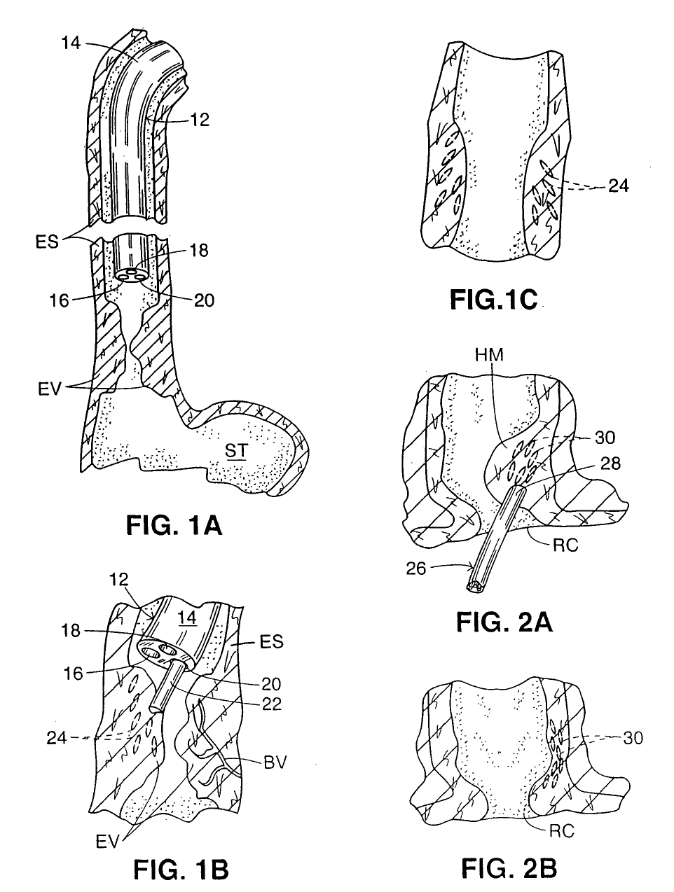

FIG. 1A shows an esophagus ES which is afflicted with varices EV at a lower end, near the stomach ST. The location and size of the varices EV are detected visually with the aid of an endoscope 12. Endoscope 12 includes a flexible insertion member 14 provided with a first light guide (not shown) having an outlet 16 for guiding electromagnetic radiation into esophagus ES to illuminate the internal tissues of the esophagus, including varices EV. Endoscope 12 is further provided with a lens 18 for focusing reflected light onto a charge-coupled device (not shown) or the input end of an optical fiber bundle (not shown).

Endoscope 12 has a biopsy channel 20 through which a tubular instrument 22 is deployed so that a distal end portion of the instrument (not separately labeled) is positionable in contact with the varices EV, as shown in FIG. 1B. Instrument 22 is operated to inject a plurality of magnetic particles 24 into the varices EV. One or more of the magnetic particles 24 are permanent magnets. Others of the magnetic particles 24 may be made of magnetizable material such as iron or steel. Upon injection of particles 24 into varices EV, magnetic attraction causes the particles to approach one another and concomitantly constrict or collapse the tissues of the varices EV. This magnetically implemented constriction entails a closure of blood vessels BV (FIG. 1B) in the varices. The closure is sufficient to induce clotting and a permanent closure of the blood vessels. Thus, the varices EV are not likely to reappear, at least not in the same location in the esophagus ES.

As illustrated in FIG. 2A, a free or distal end 28 of a tubular medical instrument 26 is inserted into a rectum RC and placed in contact with a hemorrhoid HM. The instrument 26 is operated to inject a plurality of magnetic elements 30 into the hemorrhoid HM. One or more of the magnetic elements 30 are permanent magnets. Other magnetic elements 30 may be made of magnetizable material such as iron or steel. Under the influence of magnetic attraction, elements 30 approach one another upon injection thereof into hemorrhoid HM and thus result in a constriction or internal clamping of the hemorrhoidal tissues.

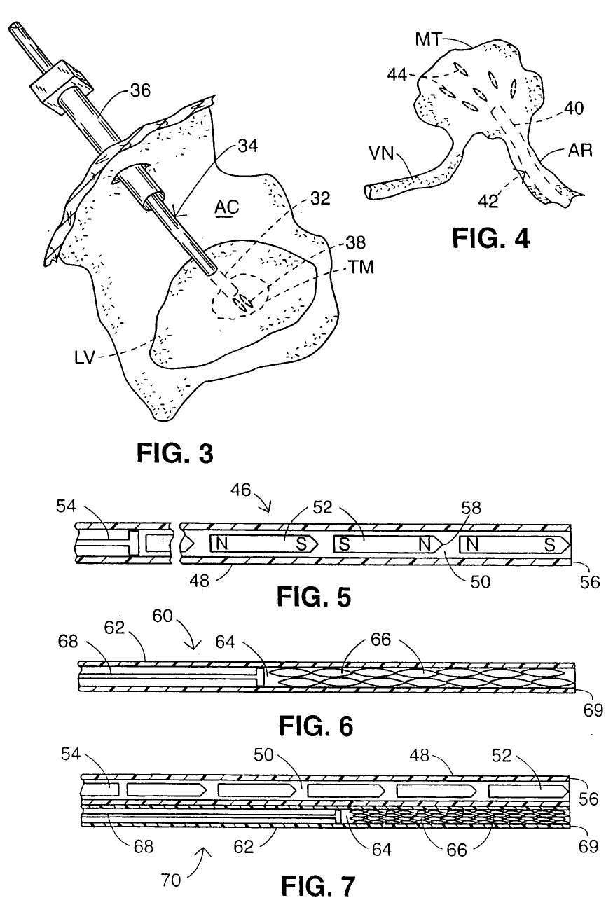

As depicted in FIG. 3, a distal end portion 32 of a tubular laparoscopic instrument 34 is inserted through a cannula or trocar sleeve 36 into an abdominal cavity AC of a patient. Distal end portion 32 of instrument 34 is further inserted into an internal organ such as the liver LV of the patient so that the distal tip of the instrument is placed into effective contact with a tumor TM inside the organ. Instrument 34 is operated to inject a plurality of magnetic elements 38 into tumor TM. As described above, injection of elements 38 into tumor TM results in a contraction of the tumor and an at least partial constriction of blood vessels (not shown) of the tumor. The constriction of the blood vessels in the tumor TM induces clotting and a permanent closure of the blood vessels. Without an adequate blood supply, the tumor TM dies.

FIG. 4 shows an alternate procedure for destroying a tumor MT having a blood supply including an artery AR and a vein VN. A distal end portion 40 of a flexible tubular medical instrument 42 in inserted through artery AR (or vein VN) into tumor MT. Then instrument 42 is operated to inject magnetic particles 44 into tumor MT, resulting in an at least partial collapse of the tumor's blood vessels and a clotting leading to tumor destruction.

The laparoscopic procedure of FIG. 3 and the intravascular procedure of FIG. 4 are performed using well-established laparoscopic and radiographic techniques. Alternatively, the deployment of laparoscopic instrument 34 and intravascular instrument 42 may be implemented under observation mediated by ultrasound. Such techniques are described in U.S. Pat. Nos. 5,871,446, 6,023,632, 6,106,463, and 6,139,499. Alternatively or additionally, the operation of instruments 34 and 42 may be robotically mediated, under remote control, as described in U.S. Pat. Nos. 5,217,003, 5,217,453, and 5,368,015.

Instruments 22, 26, 34, and 42 may take a form described now with reference to FIGS. 5-8. As depicted in FIG. 5, a medical treatment instrument 46 includes a rigid or flexible tubular member 48 with a lumen 50 carrying a plurality of permanent magnets 52. Magnets 52 are disposed end to end, with like magnetic poles (S, N) facing one another to thereby space the magnets 52 along lumen 50. A pressure applicator in the form of a plunger 54 is provided for ejecting magnets 52 from a distal tip 56 of tubular member 48. Alternative devices for the application of an ejection force to the array of magnets 52 include pumps, syringes, and other hydrostatic fluid injectors (none shown). In such a case, magnets 52 are disposed in lumen 50 in a biocompatible fluid such as saline or gel. Magnets 52 are optionally formed at a leading end with a point or edge 58 for facilitating the insertion of the magnets into organic tissues of a patient during a medical treatment procedure.

An instrument 60 shown in FIG. 6 includes a rigid or flexible tubular member 62 having a lumen 64 carrying a multiplicity of magnetizable elements 66 such a metal filings. A plunger 68 is slidably disposed in a proximal portion of lumen 64 for applying an ejection pressure to magnetizable elements 66. The function of plunger 68 may be alternatively performed by a pump, a syringe, or some other pressure application device such as a shifting sleeve magnetically linked to filings 66.

Instruments 46 and 62 (FIGS. 5 and 6) may be used successively in the same medical operation, for example, to inject one or more magnets 52 into a body of organic tissues and subsequently to inject a plurality of metal filings 66.

FIG. 7 depicts an instrument 70 representing a combination of the instruments of FIGS. 5 and 6. The same reference numerals are used in FIG. 7 to designate the same components in FIGS. 5 and 6. Tubular members 48 and 62 are connected to one another so that they extend parallel to one another. The distal tips 56 and 69 of tubular members 48 and 62 may be coplanar as shown in FIG. 7 or longitudinally spaced. Instrument 70 facilitates a single deployment procedure and simultaneous or temporally staggered ejection of magnets 52 and filings 66. Instrument 70 is particularly, but not exclusively, adapted for use in the procedures of FIGS. 2A and 3.

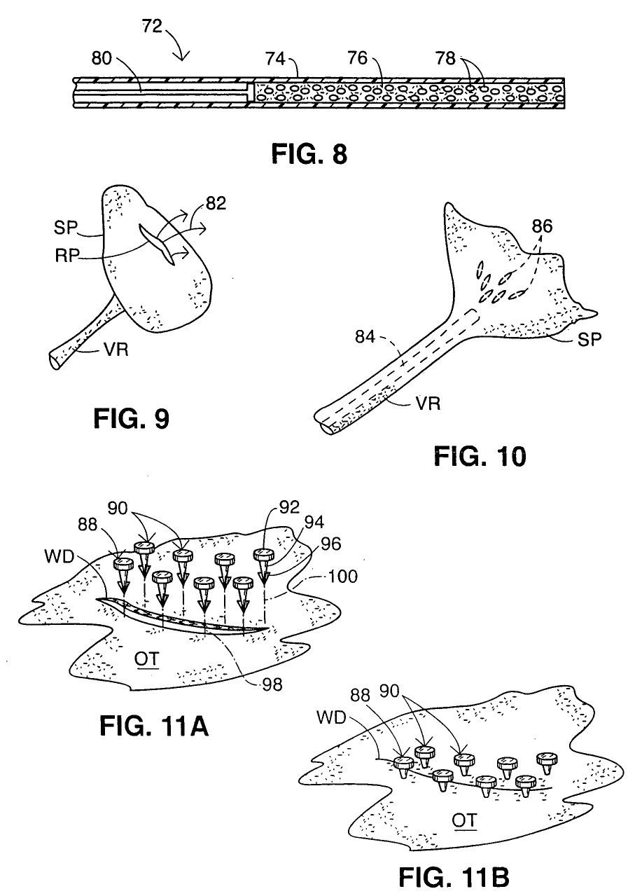

Another instrument 72 (FIG. 8) for executing a magnet injection medical procedure includes a rigid or flexible tubular member 74 carrying a fluid or gel matrix 76 in which a plurality of magnetic particles 78 are embedded. A plunger 80 or other pressure application device is operatively connected to tubular member 74 for forcibly ejecting matrix 76 and particles 78 to place the particles in a target tissue mass.

As shown in FIG. 9, an internal organ such as a spleen SP may be afflicted with a wound or rupture RP inflicted, for example, by a blunt trauma to the person of the individual patient. The rupture RP results in bleeding, indicated by arrows 82. As illustrated in FIG. 10, a minimally invasive surgical treatment of the injured organ SP entails the insertion of a distal end portion (not separately designated) of a tubular member 84 into the organ, for instance, intravascularly through a vein or artery VR supplying the organ. A plurality of magnetic elements or particles 86 are ejected from distal end of the inserted tubular member 84 into the injured organ SP. The magnetic particles 86 are injected into the injured organ SP at a location which results in a collapsing or constriction of a blood supply to the ruptured portion of the organ SP, thereby arresting the bleeding 82.

FIGS. 11A and 11B depict successive steps in a wound closure procedure utilizing two groups of magnetic tacks 88 and 90. Tacks 88 and 90 each includes a head 92 and a stem 94 provided with barbs 96 for preventing tack removal. Stems 94 of tacks 88 are inserted into organic tissues OT on one side of a wound WD, as indicated by dot-dash insertion lines 98. Similarly, stems 94 of tacks 90 are inserted into organic tissues OT on an opposite side of wound WD, as indicated by dot-dash insertion lines 100. Tacks 88 and/or 90 are permanently magnetized. Those tacks which are not magnetized are made of a magnetizable material. Magnetic attraction between tacks 88 and tacks 90 cause tacks 88 and 90 to compress the intervening tissues OT and close wound WD. Tacks 88 and 90 may be inserted in an open surgical procedure or alternatively in a minimally invasive operation using a tubular tack applicator (not shown).

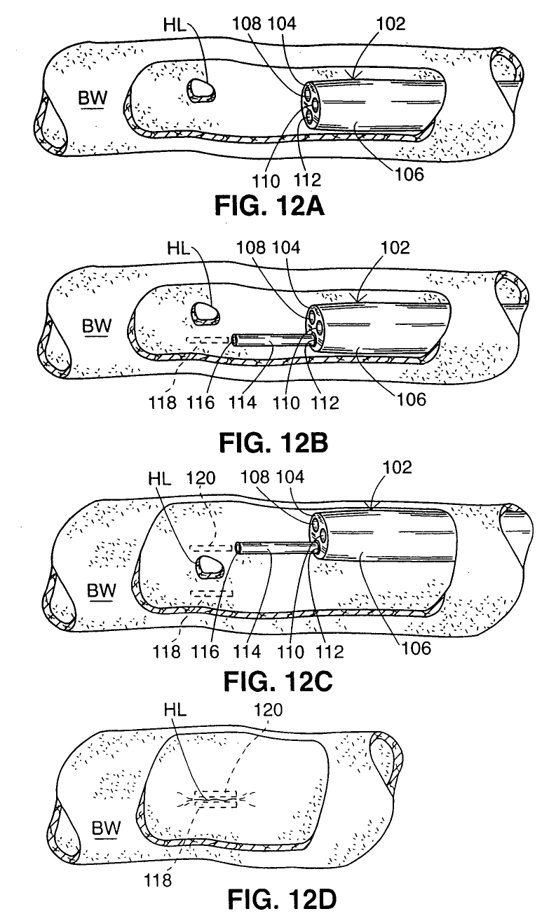

FIGS. 12A through 12D depict successive steps in an endoscopic procedure for closing a hole HL in a wall of an internal organ such as a bowel BW. As indicated in FIG. 12A, hole HL is detected via an endoscope 102 which is provided at a distal end 104 of a flexible shaft or insertion member 106 with an illumination port 108, a lens 110, and a biopsy channel mouth 112. To close hole HL, a distal end portion of a tubular instrument 114 is ejected from biopsy channel mouth 112. Upon a placement of a distal tip 116 of instrument 114 in contact with the wall of bowel BW proximate to hole HL, instrument 114 is operated to inject a magnetic element 118 into the bowel wall. Endoscope insertion member 106 is subsequently manipulated to position the distal tip 116 of instrument 114 in contact with the wall of bowel BW on an opposite side of hole HL. At that juncture, another magnetic element 120 is injected into the wall of bowel BW as shown in FIG. 12C. An attractive magnetic force between elements 118 and 120 causes them to approach one another and thereby close hole HL, as indicated in FIG. 12D.

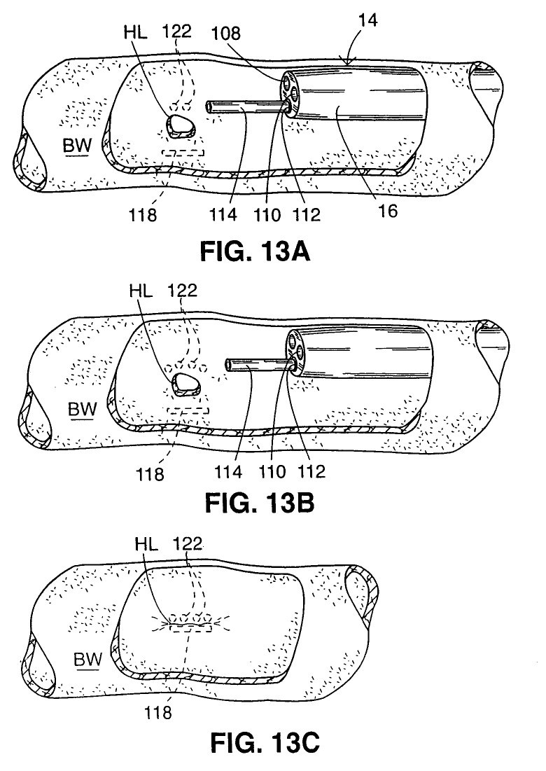

FIGS. 13A through 13C depict successive steps in a modification of the procedure of FIGS. 12A-12D, in which elongate magnetic element 120 is replaced by a group of smaller magnetic elements 122. Elements 122 are injected in sequence into the wall of bowel BW at spaced points on a side of hole HL opposite magnetic element 118. Elements 122 are substantially spherical and easily rotate inside the tissues of bowel BW, thereby facilitating registration or alignment of opposite magnetic poles on element 118 on the one hand and elements 122 on the other hand.

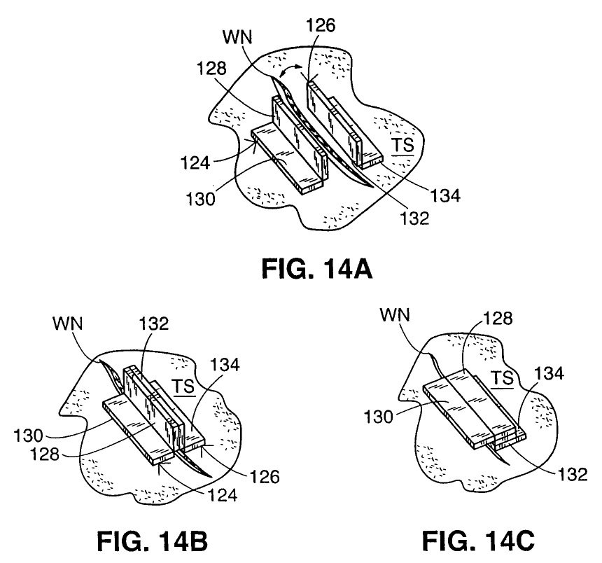

FIGS. 14A through 14C depict successive steps in a procedure for closing a wound WN utilizing a pair of closure components 124 and 126. Closure component 124 includes a magnetic plate 128 hingedly secured to an adhesive strip 130. Similarly, closure component 126 comprises a magnetic plate 132 pivotably attached to an adhesive strip 134. As indicated in FIG. 14A, adhesive strips 130 and 134 are first attached to a tissue surface TS on opposite sides of wound WN. Plates 128 and 132 are angled with respect to their respective adhesive strips 130 and 134 so that the plates face one another across wound WN. Plates 128 and 132 are magnetized so that the facing sides of the plates exhibit opposite magnetic poles. Magnetic attraction causes plates 128 and 132 to clamp to one another, as illustrated in FIG. 14B, thereby closing wound WN. Plates 128 and 132 are pivoted, as illustrated in FIG. 14C, to flatten the plates against tissue surface TS.

The various magnetic elements disclosed herein, including particles or filings 24, 38, 44, 66, 78, 86, tacks 88 and 90, elements 118, 120, 122, and plates 128 and 132, as well as adhesive strips 130 and 134, may be made of a bioabsorbable material with embedded or dispersed ferromagnetic atoms.

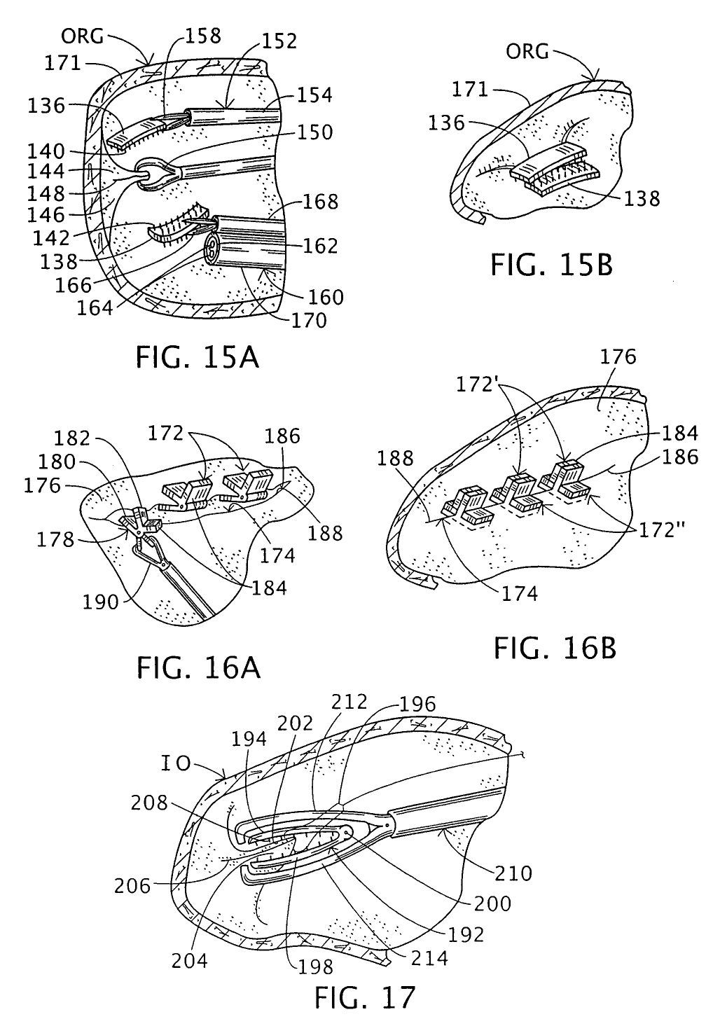

As illustrated in FIGS. 15A and 15B, a magnetic surgical closure assembly includes a pair of elongate sealing strips 136 and 138 that are made of magnetic material. At least one of the closure elements 136 and 138 is made of permanently magnetized material. The other element 136 or 138 is made of magnetizable or magnetized material. At least one and preferably both of the magnetic elements 136 and 138 are provided along a major face or surface with a plurality of coupling elements in the form of hooks, barbs, or prongs 140 or 142 that anchor the respective magnetic element 136 or 138 to respective tissues 144 or 146 of the patient along opposite sides or edges of an incision or wound 148 of the patient.

As illustrated in FIG. 15A, a surgical instrument such as a graspers or forceps 150 may be used to entrain tissues 144 and 146 and pull the tissues in a proximal direction (towards the surgeon or towards a handle end of graspers or forceps 150). This procedure draws the tissues 144 and 146 and facilitates the deployment of magnetic elements 136 and 138 to approximate and hold tissues 144 and 146 adjacent to one another and thereby close incision or wound 148.

Magnetic elements 136 and 138 may be deployed via a dedicated instrument 152 including, for instance, a tubular insertion member 154 and a forceps 158, or via an endoscope assembly 160 including (a) an endoscope insertion member 162 having optical components 164 and a manipulation instrument 166 such as a graspers or forceps inserted via a working channel 168 in endoscope insertion member 162 or (as illustrated) a sheath 170 temporarily attached to and surrounding endoscope insertion member 162. FIG. 15B shows magnetic elements 136 and 138 holding tissues 144 and 146 together to close incision or wound 148.

Magnetic elements 136 and 138 may be applied to tissues 144 and 146 during open surgery or laparoscopic surgery or trans-organ surgery utilizing the techniques of U.S. Pat. Nos. 5,297,536 and 5,458,131. In laparoscopic or trans-organ surgery, magnetic elements may be deployed via an endoscope working channel 168 as discussed above. In trans-organ surgery, tissues 144 and 146 may be portions of a wall 171 of an internal organ ORG such as the stomach, vagina, urinary bladder or colon, through which a surgical operation is performed in the abdominal cavity (not designated).

Magnetic elements 136 and 138 clamp tissues 144 and 146 together for a sufficient time to enable healing. Where tissues 144 and 146 are portions of a wall 171 of an internal organ ORG such as the stomach, vagina, urinary bladder or colon, an internal surface 173 of the organ wall 171 constitutes a mucosal layer, which is difficult to mend to itself. Consequently, wall 171 is invaginated at incision or wound 148 so that external surfaces (not designated) of tissues 144 and 146 are disposed in contact with one another.

As illustrated in FIGS. 16A and 16B, a plurality of approximation or closure devices 172 are used to close a wound or incision 174 in internal tissues 176. Each approximation or closure device 172 includes a spring-loaded clamp 178 with jaws 180 and 182. A magnetic plate 184 is connected to at least one of the jaws 180 and 182. The magnetic plate 184 is made of a magnetic material, either a permanent magnetic material or a magnetizable material. A first set of approximation or closure devices 172′ are applied to one edge or lip 186 of incision or wound 174 while a second set of approximation or closure devices 172″ are applied to an opposite edge or lip 188 of incision or wound 174 so that the magnetic plates 184 of devices 172′ are disposed adjacent the magnetic plates 184 of respective devices 172″ so as to magnetically couple devices 172′ to devices 172″ and thus close wound or incision 174 (FIG. 16B). A forceps 190 or other instrument is used to apply clamps 178 of devices 172′ and 172″ to incision lips 186 and 188.

As illustrated in FIG. 17, a wound closure device 192 comprises a clamp with jaws 194 and 198 hinged to one another at 200. Wound closure device 192 may be spring loaded so that jaws 194 and 198 are biased to close the device about internal tissues 202 and 204 on opposite sides of an incision or wound 206, thereby closing the incision or wound. Jaws 194 and 198 are optionally provided along inner, facing surfaces with hooks, barbs or prongs 208 for enhancing a coupling of the jaws to tissues 202 and 204 during a surgical closure operation. Device 192 may be applied via a grasping or forceps instrument 210 having jaws 212 and 214, as disclosed in U.S. Pat. Nos. 5,015,249, 5,049,153, and 5,156,609. A suture 196 may be temporarily inserted through tissues or lips 202 and 204 to draw the tissues or lips 202 and 204 together and to invaginate the tissues along wound or incision 206 to avoid approximation of mucosal tissues, in the event that tissues 202 and 204 are parts of an internal organ IO such as a stomach, urinary bladder, vagina, or colon, pursuant to the teachings of U.S. Pat. Nos. 5,297,536 and 5,458,131.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A medical treatment method comprising:
placing at least one first magnetic element in contact with first organic tissues of a patient on one side of a feature to be closed or collapsed;
placing at least one second magnetic element in contact with second organic tissues of the patient on an opposite side of the feature to be closed or collapsed; and
by virtue of a magnetic attraction between said first magnetic element and said second magnetic element, holding said first and said second organic tissues of the patient together to close or collapse said feature,
the placing of said first magnetic element in contact with said first organic tissues including placing said first magnetic element into contact with and along an external surface of said first organic tissues, such that said first magnetic element remains outside of said first organic tissues,
the placing of said second magnetic element in contact with said second organic tissues including placing said second magnetic element into contact with and along an external surface of said second organic tissues, such that said second magnetic element remains outside of said second organic tissues.

2. The method defined in claim 1 wherein at least one of said first magnetic element and said second magnetic element takes the form of an elongate strip, the placing of one of said first magnetic element and said second magnetic element in contact with one of said first and said second organic tissues including placing said strip along said one of said first organic tissues and said second organic tissues.

3. The method defined in claim 2 wherein said elongate strip is provided along one surface with a plurality of coupling elements taken from the group consisting of barbs, hooks, and prongs, further comprising inserting said coupling elements into said one of said first organic tissues and said second organic tissues to anchor said strip to said one of said first organic tissues and said second organic tissues.

4. The method defined in claim 1 wherein at least one of said first magnetic element and said second magnetic element includes a clip, the placing of one of said first magnetic element and said second magnetic element in contact with organic tissues including actuating said clip to grasp one of said first organic tissues and said second organic tissues.

5. The method defined in claim 4 wherein said feature is a wound or incision having a pair of edges or lips, said clip having a pair of clamping elements taken from the group consisting of legs, prongs, and arms, the actuating of said clip includes inserting one of said clamping elements of said clip through said wound or incision.

6. The method defined in claim 1 wherein said first magnetic element and said second magnetic element are parts of a clamp, said first magnetic element and said second magnetic element being hingedly connected to one another, further comprising pivoting said first magnetic element and said second magnetic element relative to one another to close said clamp on said first organic tissues and said second organic tissues, thereby closing said feature.

7. The method defined in claim 1 wherein at least one of said first magnetic element and said second magnetic element is a permanent magnet.

8. The method defined in claim 1 wherein the placing of said first magnetic element and said second magnetic element includes inserting an endoscope into the patient and ejecting said first magnetic element and said second magnetic element from a working channel of said endoscope.

9. A medical treatment device comprising an elongate strip of magnetic material provided along a major face or surface with a plurality of coupling elements taken from the group consisting of barbs, prongs and hooks.

10. The treatment device defined in claim 9 wherein said strip is one of two wound-closure strips provided in a medical treatment kit, the other of said wound-closure strips being made of magnetic material, the other of said wound-closure strips being provided along a major face or surface with a plurality of coupling elements taken from the group consisting of barbs, prongs and hooks.

11. A medical treatment device comprising a clamp and a magnetic element attached to said clamp.

12. The treatment device defined in claim 11 wherein said clamp includes a pair of jaws hingedly secured to one another, said magnetic element being connected to at least one of said jaws.

13. The treatment device defined in claim 12 wherein said magnetic element is a plate shaped component.

14. A medical treatment kit comprising:
at least one first magnetic element disposable in contact with first organic tissues of a patient on one side of a feature to be closed or collapsed; and
at least one second magnetic element disposable in contact with second organic tissues of the patient on an opposite side of the feature to be closed or collapsed,
said first magnetic element and said second magnetic element having a magnetic attraction to one another sufficient to hold said first and said second organic tissues together to close or collapse said feature,
said first magnetic element being disposable in contact with and along an external surface of said first organic tissues, such that said first magnetic element remains outside of said first organic tissues,
said second magnetic element being disposable in contact with and along an external surface of said second organic tissues, such that said second magnetic element remains outside of said second organic tissues.

15. The treatment kit defined in claim 14 wherein at least one of said first magnetic element and said second magnetic element takes the form of an elongate strip.

16. The treatment kit defined in claim 15 wherein said elongate strip is provided along one surface with a plurality of coupling elements taken from the group consisting of barbs, hooks, and prongs.

17. The treatment kit defined in claim 14 wherein at least one of said first magnetic element and said second magnetic element includes a clip actuatable to grasp one of said first organic tissues and said second organic tissues.

18. The treatment kit defined in claim 14 wherein said clip has a pair of clamping elements taken from the group consisting of legs, prongs, and arms insertable through said wound or incision.

19. The treatment kit defined in claim 14 wherein said first magnetic element and said second magnetic element are parts of a clamp, said first magnetic element and said second magnetic element being hingedly connected to one another.

20. The treatment kit defined in claim 14 wherein at least one of said first magnetic element and said second magnetic element is a permanent magnet.

21. The treatment kit defined in claim 14 wherein said first magnetic element and said second magnetic element are ejectable from a working channel of said endoscope.
